SEQUENCE LISTING (5 sequences)

SEQ ID NO: 1 - protein, 25 aa, Artificial sequence
  Amino acid sequence of poly(Glu,Tyr) peputide
EEEEYEEEEYEEEEYEEEEYEEEEY

SEQ ID NO: 2 - DNA, 75 bp, Artificial sequence
  Designed DNA based on amino acid sequence of poly(Glu,Tyr) peptide
gaggaggaggagtacgaagaagaagaatatgaagaagaagaatatgaagaagaagaatatgaggaggaagagtac

SEQ ID NO: 3 - DNA, 25 bp, Artificial sequence
  Designed DNA based on nucleotide sequence of poly(Glu,Tyr) peptide
ccggggatccgaggaggaggagtac

SEQ ID NO: 4 - DNA, 27 bp, Artificial sequence
  Designed DNA based on nucleotide sequence of poly(Glu,Tyr) peptide
cccggggaattcgtactcttcttcctc

SEQ ID NO: 5 - protein, 263 aa, Artificial sequence
  Amino acid sequence of GST-poly(Glu,Tyr) fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSEEEEYEEEEYEEEEYEEEEYEEEEYEFPGRLERPHRD

(19) United States
(12) Patent Application Publication
(10) Pub. No.: US 2008/0003630 A1
(43) Pub. Date: Jan. 3, 2008

(54) Tyrosine kinse substrate

(75) Inventors: Michitaka Notoya, Kobe (JP); Tomoko Ohyama, Akashi (JP); Tomokazu Yoshida, Kobe (JP); Hideki Ishihara, Miki (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 11/819,236

(22) Filed: Jun. 26, 2007

(30) Foreign Application Priority Data
Jun. 30, 2006 (JP) JP 2006-180536

(51) Int. Cl. C12Q 1/48; C07K 14/00

(52) U.S. Cl. 435/15; 530/350

(57) ABSTRACT

A tyrosine kinase substrate is described. The tyrosine kinase substrate comprises a fusion protein which comprises a protein for labeling and a specific peptide fused with the protein for labeling. The specific peptide has an amino acid sequence including a glutamic acid residue and a tyrosine residue. The tyrosine kinase substrate is capable of being phosphorylated by a plurality of kinds of tyrosine kinases.

### BACKGROUND OF THE INVENTION

1. Field of the Invention

The present invention relates to a tyrosine kinase substrate, more particularly to a tyrosine kinase substrate which can be phosphorylated by a plurality of kinds of tyrosine kinases. Also, the present invention relates to a method for measuring a tyrosine kinase using the substrate.

2. Description of the Related Art

A tyrosine kinase is an enzyme which specifically phosphorylates a tyrosine residue of a protein or a peptide. A tyrosine kinase is present in a cell membrane or a cytoplasm, and plays an important role in differentiation and proliferation of a cell. In addition, it is known that abnormality of an expression amount or an enzyme activity of a tyrosine kinase cause canceration of a cell. For example, it is reported that an insulin-like growth factor receptor (IGFR) is overexpressed in a tumor cell. In addition, it is known that HER1 among a human epithelial growth factor receptor (HER) is enhanced in the activity mainly in a lung cancer. It is known that HER2 is enhanced in the activity mainly in a breast cancer. Abnormality of an expression amount or an enzyme activity of a tyrosine kinase in a cell can be detected by measuring an activity value of the tyrosine kinase present in a cell membrane or a cytoplasm. Therefore, measurement of the activity of a tyrosine kinase is essential for elucidating the canceration mechanism of a cell.

There is a method for measuring the activity of a tyrosine kinase by mixing a tyrosine kinase, a substrate and ATP, and detecting a phosphorylated substrate. Such method is described, for example, in a literature by Norio Sasaki et al. (Norio Sasaki et al., 1985, The Journal of Biological Chemistry, Vol. 260, No. 17, 9793-9804). In the literature, upon measurement of the activity of a tyrosine kinase, a commercially available synthetic peptide is used as a substrate. The synthetic peptide is a peptide synthesized so that an amino acid sequence including a glutamic acid residue (hereinafter, abbreviated as Glu) and a tyrosine residue (hereinafter, abbreviated as Tyr) is repeated plural times. It is known that the synthetic peptide is phosphorylated by a plurality of kinds of tyrosine kinases. Therefore, by using such synthetic peptide in measurement of the activity of a tyrosine kinase, it becomes possible to measure the activity of a plurality of tyrosine kinases.

However, the aforementioned commercially available synthetic peptide has a special amino acid sequence in which a sequence consisting of a few amino acids comprising Glu and Tyr is repeated. For this reason, when the synthetic peptide is chemically synthesized, it is difficult to control a length of a peptide, and the commercially available synthetic peptide is not of a uniform molecular weight. Therefore, the commercially available synthetic peptide cannot be detected as a single band in electrophoresis such as SDS-PAGE. In addition, when the synthetic peptide having such amino acid sequence is applied to Western blotting or slot blotting, the synthetic peptide exhibits behavior different from that of a normal protein, and the synthetic peptide cannot be transferred to or adsorbed onto a membrane well in some cases.

From the foregoing, the commercially available synthetic peptide cannot be applied to a method for measuring a tyrosine kinase, comprising a step of electrophoresis, Western blotting or slot blotting.

### SUMMARY OF THE INVENTION

The scope of the present invention is defined solely by the appended claims, and is not affected to any degree by the statements within this summary.

An object of the present invention is to provide a substrate which can be phosphorylated by a plurality of kinds of tyrosine kinases, and can be applied to a method for measuring a tyrosine kinase, comprising a step of electrophoresis, Western blotting or slot blotting. Another object of the present invention is to provide a method for measuring a tyrosine kinase using the substrate.

A first aspect of the present invention is a tyrosine kinase substrate comprising a fusion protein which comprises a protein for labeling and a specific peptide fused with the protein for labeling, wherein the specific peptide has an amino acid sequence including a glutamic acid residue and a tyrosine residue.

A second aspect of the present invention is a method for measuring the activity of a tyrosine kinase, comprising steps of:

contacting the tyrosine kinase with a tyrosine kinase substrate and a phosphate group donor so that phosphorylate the substrate, wherein the substrate comprises a fusion protein which comprises a protein for labeling and a specific peptide fused with the protein for labeling, and the specific peptide has an amino acid sequence including a glutamic acid residue and a tyrosine residue;

detecting the phosphorylated substrate; and

measuring the activity of the tyrosine kinase based on the result of detection of the phosphorylated substrate.

### BRIEF DESCRIPTION OF THE DRAWINGS

FIG. 1 shows the result of Western blotting in Example 2.

FIG. 2 shows the result of Western blotting in Example 3.

FIG. 3 is a graph showing the result of ELISA in Example 4.

FIG. 4 is a graph showing the result of ELISA in Example 5.

FIG. 5 shows the result obtained by CBB-staining a membrane of slot blotting in Example 6.

### DESCRIPTION OF THE PREFERRED EMBODIMENTS

A tyrosine kinase substrate which is one embodiment of the present invention is a fusion protein of a specific peptide and a protein for labeling. The specific peptide includes an amino acid sequence comprising Glu and Tyr (hereinafter, referred to as (Glu, Tyr) peptide). The fusion protein obtained by fusing the (Glu, Tyr) peptide with the protein can be applied to measurement of the activity of a tyrosine kinase (e.g. measurement of the activity of a tyrosine kinase comprising electrophoresis, Western blotting or slot blotting) to which the conventional commercially available synthetic peptide cannot be applied.

The amino acid sequence of the (Glu, Tyr) peptide is not particularly limited as far as it comprises Glu and Tyr, and Tyr contained therein can be phosphorylated by a plurality of kinds of tyrosine kinases. Examples of such amino acid sequence include amino acid sequences of synthetic peptides used as a substrate for a tyrosine kinase in the literature by Norio Sasaki et al., a literature by Sergei Braun et al. (Sergei Braun et al., 1984, The Journal of Biological Chemistry, Vol. 259, No. 4, 2051-2054) and a literature by M. Abdel-Ghany et al. (M. Abdel-Ghany et al., 1990, Proceeding of The National Academy of Science, Vol. 87, 7061-7065). Specifically, there can be exemplified:

an amino acid sequence in which a sequence consisting of four Glu's and one Tyr is repeated two or more rimes (hereinafter, referred to as amino acid sequence (i)),

an amino acid sequence in which a sequence consisting of one Glu and one Tyr is repeated two or more times (hereinafter, referred to as amino acid sequence (ii)),

an amino acid sequence in which a sequence consisting of six Glu's, one Tyr and three alanine residues (hereinafter, abbreviate as Ala) is repeated two or more times (hereinafter, referred to as amino acid sequence (iii)),

an amino acid sequence in which a sequence consisting of one Glu, one Tyr and one Ala is repeated two or more times (hereinafter, referred as to amino acid sequence (iv)),

an amino acid sequence in which a sequence consisting of two Glu's, one Tyr, six Ala's and five lysine residues (hereinafter, abbreviated as Lys) is repeated two or more times (hereinafter, referred to amino acid sequence (v)).

A literature by Tony Hunter (Tony Hunter, 1982, The Journal of Biological Chemistry, Vol. 257, No. 9, 4843-4848) reports that an acidic amino acid residue is important for phosphorylation of Tyr with a tyrosine kinase. Thereby, among the amino acid sequences (i) to (v), particularly, the amino acid sequence (i) and the amino acid sequence (iii) containing a large amount of Glu which is an acidic amino acid residue are preferable.

Generally, an enzyme recognizes a steric structure of a particular substrate and reacts with it. However, since amino acid sequences of the (Glu, Tyr) peptides are special amino acid sequences comprising Glu and Tyr, they are low in specificity for kinds of tyrosine kinases, and can be phosphorylated by a plurality of kinds of tyrosine kinases. Therefore, a fusion protein comprising the (Glu, Tyr) peptide can measure the activity of a tyrosine kinase, regardless of a kind of the tyrosine kinase contained in a sample.

The fusion protein comprises the (Glu, Tyr) peptide consisting of the amino acid sequence. Therefore, the fusion protein can be used as a substrate which can be phosphorylated by a plurality of kinds of tyrosine kinases. Examples of a tyrosine kinase which phosphorylates the fusion protein include growth factor receptors such as an insulin receptor (IR), an insulin-like growth factor receptor (IGFR), a platelet-derived growth factor receptor (PDGF), a fibroblast growth factor receptor (FGFR), a human epithelial growth factor receptor (HER), a vascular endothelial growth factor receptor (VEGFR), which are receptor tyrosine kinases. Other examples include Src, Frk, Btk, Csk, Abl, Zap70, Fes, Fps, Fak, Jak and Ack which are non-receptor tyrosine kinases.

The protein for labeling is not particularly limited as far as it is a protein or a fragment of a protein which can be applied to electrophoresis, Western blotting or slot blotting, and dose not inhibit phosphorylation of a substrate with a tyrosine kinase. It is preferable that the protein for labeling is a protein or a fragment of a protein having a molecular weight of at least 10 kDa so that it can be applied to electrophoresis, Western blotting or slot blotting. In addition, when a molecular weight of a protein for labeling is too large, an efficiency of phosphorylation of the fusion protein with a tyrosine kinase is reduced, and an efficiency of transfer of the fusion protein onto a membrane in Western blotting is reduced. Therefore, it is preferable that a protein for labeling is a protein or a fragment of a protein having a molecular weight of not more than 100 kDa. Examples of the protein for labeling which satisfies such condition include glutathione-S-transferase (hereinafter, abbreviated as GST), protein A, thioredoxin, cellulose binding domain, β-lactamase, β-galactosidase, luciferase, heat shock protein, fibronectin partial peptide, green fluorescent protein, yellow fluorescent protein, cyan fluorescent protein, blue fluorescent protein, red fluorescent protein, maltose binding protein, immunoglobulin G, avidin, streptavidin, and protein G.

From a viewpoint of easy expression and purification of the fusion protein, it is preferable to use, for example, a protein used as an affinity tag, or a fragment thereof, as a protein for labeling. The “affinity tag” referred herein has a group or a part which can bind to a specific binding partner with a high affinity. The affinity tag is generally used in purification of a protein or a peptide. It is preferable that the affinity tag used as the protein for labeling has a molecular weight of at least 10 kDa for the same reason as the aforementioned one. It is preferable that the affinity tag used as the protein for labeling has a molecular weight of not more than 100 kDa for the same reason as the aforementioned one. Examples of the affinity tag satisfying such condition include glutathione-S-transferase (hereinafter, abbreviated as GST), maltose-binding protein, avidin, streptavidin, thioredoxin, cellulose binding domain, immunoglobulin G, protein A, and protein G. Among them, particularly, GST, maltose binding protein, avidin, and streptavidin are preferable.

The fusion protein can be easily prepared by a known genetic engineering procedure. For example, a DNA fragment encoding the (Glu, Tyr) peptide and a DNA fragment encoding the protein for labeling are obtained, each DNA fragment is digested with a restriction enzyme, and the DNA fragments are connected and incorporated into a suitable vector. A vector used is not particularly limited as far as it can express the fusion protein of the (Glu, Tyr) peptide and the protein for labeling in a cell into which the vector is introduced. It is convenient to use a vector into which a DNA sequence encoding the protein for labeling has been incorporated inadvance. For example, when GST is utilized as the protein for labeling, the fusion protein of the (Glu, Tyr) peptide and the protein for labeling can be easily expressed by incorporating a DNA fragment encoding the (Glu, Tyr) peptide into an expression vector for a GST fusion protein.

An amino acid sequence of the fusion protein may comprise one or more amino acid residues which do not derive from the (Glu, Tyr) peptide and the protein for labeling. For example, when a DNA fragment of the (Glu, Tyr) peptide is amplified by PCR, a restriction enzyme site may be inserted into a 5′-terminus of a primer in order to enhance the amplification efficiency and facilitate vector construction thereafter. In that case, one or more amino acid residues may be inserted between the protein for labeling and the (Glu, Tyr) peptide. Alternatively, depending on an insertion part of an expression vector into which a DNA fragment is incorporated, one or more amino acid residues may be comprised in the amino acid sequence of the fusion protein. Even such fusion protein can be used as far as it reacts with a tyrosine kinase as a substrate for a plurality of kinds of tyrosine kinases.

The fusion protein can be applied to measurement of the activity of a tyrosine kinase as a substrate for a tyrosine kinase. Examples of such method include a method comprising a step of phosphorylating the fusion protein by contacting the fusion protein, a tyrosine kinase and a phosphate group donor, a step of detecting the phosphorylated fusion protein, and a step of determining the activity of a tyrosine kinase based on the detection result.

Herein, the “contact” includes mixing. And, a step of phosphorylating the fusion protein can be performed, for example, by mixing the fusion protein, a tyrosine kinase and a phosphate group donor. Examples of the phosphate group donor include adenosine triphosphate (ATP), adenosine 5′-O-(3-thiotriphosphate) (ATP-γS), 32P-labeled adenosine 5′-O-(3-triphosphate) (γ-[32P]-ATP), adenosine diphosphate (ADP), adenosine monophosphate (AMP) and the like.

As described above, the commercially available synthetic peptide does not have a uniform molecular weight, and may exhibit behavior different from that of a normal protein. For this reason, the commercially available synthetic peptide could not have been applied to measurement of the activity of a tyrosine kinase comprising a step of electrophoresis, Western blotting or slot blotting. On the other hand, since the fusion protein is prepared by a genetic engineering procedure, a molecular weight thereof becomes uniform. Further, since the fusion protein is a fusion protein in which the (Glu, Tyr) peptide is fused with the protein for labeling, it exhibits the same behavior as that of a normal protein. Thereby, it becomes possible to apply the fusion protein to measurement of the activity of a tyrosine kinase comprising electrophoresis, Western blotting or slot blotting.

For example, when the fusion protein is applied to Western blotting, phosphorylation of the fusion protein can be detected by the following method. First, the phosphorylated fusion protein and other proteins are separated with a membrane. Then, an antibody recognizing a phosphorylated fusion protein (hereinafter, referred to as phosphorylated fusion protein-specific antibody) is bound to the phosphorylated fusion protein on the membrane. Further, a secondary antibody having a fluorescent substance is bound to the phosphorylated fusion protein-specific antibody bound to the phosphorylated fusion protein. And, by detecting fluorescent light of the secondary antibody bound to the phosphorylated fused protein-specific antibody, phosphorylation of the fusion protein can be detected. When the phosphorylated fusion protein is separated from other proteins in advance, phosphorylation of the fusion protein can also be detected using a slot blotting method in place of Western blotting. An enzyme may be used in place of the fluorescent substance. In this case, a substrate for an enzyme is added to the enzyme possessed by a secondary antibody to perform a color developing reaction, and color development generated by the reaction may be detected. Alternatively, a solution containing a phosphorylated fusion protein is put into a tube, a phosphorylated fusion protein-specific antibody having a fluorescent substance is added thereto to bind to a phosphorylated fusion protein, and a fluorescent intensity is measured, thereby, phosphorylation of the fusion protein may be detected. Further, when the fusion protein is applied to electrophoresis, the fusion protein phosphorylated by 32P can be detected by phosphorylating the fusion protein using radiolabeled ATP ([γ-32P]ATP), separating the fusion protein phosphorylated by 32P by electrophoresis, and analyzing this by an imaging analyzer.

The fusion protein can be used in methods other than those described above. Specifically, examples include a solid-phase enzyme linked immunosorbent assay (hereinafter, referred to as ELISA method), and a radioimmunoassay (hereinafter, referred to as RIA method). The ELISA method includes a direct adsorption method and a sandwich method. In the direct adsorption method, a phosphorylated fusion protein is adsorbed onto a surface of a solid phase, and a phosphorylated fusion protein-specific antibody having an enzyme is added thereto to bind to the phosphorylated fusion protein. Then, a substrate for the enzyme is added to the enzyme possessed by the phosphorylated fusion protein-specific antibody to perform a color developing reaction, and color development generated by this reaction may be detected. In the sandwich method, a phosphorylated fusion protein-specific antibody is bound to a solid phase (hereinafter, referred to as solid phased antibody), and a phosphorylated fusion protein is added thereto to bind to the solid phased antibody. Then, a phosphorylated fusion protein-specific antibody having an enzyme (hereinafter, referred to as labeled antibody) is added thereto to bind to the phosphorylated fusion protein. A substrate for an enzyme is added to the enzyme possessed by the labeled antibody to perform a color developing reaction, and color development generated by this reaction may be detected. For example, when the enzyme possessed by the labeled antibody is alkaline phosphatase, as a substrate, a mixed solution of nitrotetrazolium blue chloride (NBT) and 5-bromo-4-chloro-3-indoxylphosphate (BCIP) may be used to perform a reaction. When the enzyme possessed by the labeled antibody is peroxidase, as a substrate, diaminobenzidine (DAB) may be used to perform a reaction. When the sandwich method is used, the solid phased antibody and the labeled antibody are preferably bound to different sites of the phosphorylated fusion protein. That is, it is preferable that there are a plurality of antibody binding sites in the phosphorylated fusion protein, or two kinds of antibodies used recognize different antigen determinants of the phosphorylated fusion protein. When a signal generating substance is a radioactive isotope, phosphorylation of the fusion protein can be detected by radioimmunoassay (hereinafter, referred to as RIA). Specifically, a phosphorylated fusion protein-specific antibody having a radioactive isotope is bound to a phosphorylated fusion protein, and radiation can be measured by a scintillation counter or the like to detect phosphorylation of the fusion protein.

A substrate for a tyrosine kinase of the present invention will be more specifically explained below based on Examples. The present invention is not limited to these Examples.

### EXAMPLE 1

Preparation of Tyrosine Kinase Substrate

A fusion protein of a peptide (hereinafter, referred to as poly (Glu, Tyr) peptide) and a GST protein was prepared. The poly (Glu, Tyr) peptide consists of an amino acid sequence (SEQ ID No.: 1) in which a sequence consisting of four glutamic acid residues and one tyrosine residue is repeated five times. This fusion protein was used as a substrate which can be phosphorylated by a plurality of kinds of tyrosine kinases. Hereinafter, this fusion protein is referred to as GST poly (Glu, Tyr) fusion protein.

The GST poly (Glu, Tyr) fusion protein was prepared by the following method. First, PCR was performed using a DNA (SEQ ID No.: 2) encoding an amino acid sequence (SEQ ID No.: 1) of the poly (Glu, Tyr) peptide, a sense primer (SEQ ID No.: 3) designed based on a nucleotide sequence of this DNA, an antisense primer (SEQ ID No.: 4), and KOD plus DNA polymerase (TOYOBO., LTD.). The amplification product (hereinafter, referred to as poly (Glu, Tyr) DNA) was obtained by PCR. The poly (Glu, Tyr) DNA and pGEX-4T3, which is a plasmid vector for expressing a GST fusion protein (GE Health Care Bio-Sciences), were treated with a restriction enzyme (BamH1 and EcoR1). The poly (Glu, Tyr) DNA was incorporated into the pGEX-4T3 to prepare a recombinant plasmid. This recombinant plasmid was transformed into Escherichia coli JM109, and this Escherichia coli was cultured in a liquid medium (LB medium) until an absorbance (600 nm) of the culturing solution became 0.6. To this cultured Escherichia coli was added 1 mM IPTG (concentration in culturing solution), followed by culturing for 4 hours, to induce protein expression. Then, Escherichia coli was lysed, and expressed GST-poly (Glu, Tyr) fusion protein was collected by using Glutathione Sepharose 4B (GE Health Care Bio-Sciences). An amino acid sequence of this GST-poly (Glu, Tyr) fusion protein is shown in SEQ ID No.: 5.

### EXAMPLE 2

Detection of Phosphorylation of Fusion Protein by Western Blotting using Intracellular Domain of Receptor Tyrosine Kinase

Herein, using an intracellular domain (ICD) of a commercially available receptor tyrosine kinase, the GST-poly (Glu, Tyr) fusion protein prepared in Example 1 was phosphorylated. Then, the phosphorylated GST-poly (Glu, Tyr) fusion protein was detected by Western blotting. A receptor tyrosine kinase is composed of an extracellular domain, a transmembrane domain, and an intracellular domain, and a site exhibiting the activity of a tyrosine kinase is present in the intracellular domain.

1. Method of Preparing Reaction Sample

50 μl of a buffer 1 (containing 20 mM HEPES pH 7.4, 10 mM MnCl₂, 1% NP40, 1 mM DTT, 0.2% protease inhibitor (hereinafter, referred to as PI), 10% glycerol, 200 μM Na₃VO₄ and 50 mM NaF) and 0.5 pmol of ICD of a commercially available receptor tyrosine kinase were mixed, and The mixture was used as a sample for a reaction. In the present Example, ICDs of a PDGF Receptor β Kinase (hereinafter, referred to as PDGFR-β), a VEGF Receptor 1 Kinase (hereinafter, referred to as VEGFR1), a VEGF Receptor 2 Kinase (hereinafter, referred to as VEGFR2), an EGF Receptor 1 Kinase (hereinafter, referred to as HER1), an ErbB2 Kinase (hereinafter, referred to as HER2), an ErbB4 Kinase (hereinafter, referred to as HER4), and an IGF-1 Receptor Kinase (IGF1R) (all from Cell Signaling Technology) were used. A mixture of the buffer 1 and PDGFR-β is designated as a reaction sample i, a mixture of the buffer 1 and VEGFR1 is designated as a reaction sample ii, a mixture of the buffer 1 and VEGFR2 is designated as a reaction sample iii, a mixture of the buffer 1 and HER1 is designated as a reaction sample iv, a mixture of the buffer 1 and HER2 is designated as a reaction sample v, a mixture of the buffer 1 and HER3 is designated as a reaction sample vi, and a mixture of the buffer 1 and IGF1R is designated as a reaction sample vii.

2. Enzyme Reaction

25 μl of the reaction sample i and 25 μl of a substrate solution 1 (containing 20 mM HEPES pH 7.4, 10 mM MnCl₂, 1 mM DTT, 1% NP40, 0.2% PI, 10% glycerol, 200 μM Na₃VO₄, 50 mM NaF, 40 μM ATP, and 5 μg GST-poly (Glu, Tyr) fusion protein) prepared in Example 1 were mixed, and incubated at 25° C. for 60 minutes. To this reaction solution was added 25 μl of an SDS samplebuffer pH 6.8 (containing 200 mM Tris, 40% glycerol, 8% SDS, and 10% 2-mercaptoethanol), and this was boiled at 100° C. for 5 minutes to stop an enzyme reaction. The thus prepared solution is designated as an SDS sample i (+). Similarly, SDS samples ii (+) to vii (+) were prepared from the reaction samples ii to vii.

Separately, 25 μl of a reaction sample i, and 25 μl of a substrate solution 2 (containing 20 mM HEPES pH 7.4, 10 mM MnCl₂, 1 mM DTT, 1% NP40, 0.2% PI, 10% glycerol, 200 μM Na₃VO₄, 50 mM NaF, and 5 μg GST-poly (Glu, Tyr) fusion protein) were mixed, and incubated at 25° C. for 60 minutes. To this reaction solution was added 25 μl of an SDS sample buffer pH 6.8 (containing 200 mM Tris, 40% glycerol, 8% SDS, and 10% 2-mercaptoethanol), and this was boiled at 100° C. for 5 minutes to stop an enzyme reaction. The thus prepared solution is designated as an SDS sample i (−). Similarly, SDS samples ii (−) to vii (−) were prepared from the reaction samples ii to vii.

The substrate solution 2 is the same as the substrate solution 1 except that ATP is not contained. In addition, the SDS samples i (−) to vii (−) were used as negative controls of the SDS samples i (+) to vii (+).

3. Detection of Phosphorylated Fusion Protein by Western Blotting

A protein contained in the SDS samples i (+) to vii (+) and the SDS samples i (−) to vii (−) were separated by SDS-PAGE. First, 15 μl of each SDS sample was loaded into separate wells of a polyacrylamide gel (PAG mini “first” 4/20 (13W) (Daiichi Pharmaceutical Co., Ltd.)), and subjected to electrophoresis at 25 mA for 70 minutes using an electrophoretic vessel (cassette electrophoretic vessel “first” DPE-1020 (mini duplicate) (Daiichi Pharmaceutical Co., Ltd.)). A protein separated by electrophoresis was transferred from a polyacrylamide gel to a polyvinylidene fluoride (PVDF) membrane (Immobilon-FL 0.45 μm pore size (Millipore)) by applying a voltage of 100 V using a mini transblotting cell (Bio-Rad) for 1 hour. This PVDF membrane was blocked with a 4% Block Ace (Dainippon Sumitomo Pharma Co., Ltd.) solution for 60 minutes. The blocked PVDF membrane was shaken in 2 ml of a primary antibody solution (containing 0.4% Block Ace and 0.5 μg/ml Anti-Phosphotyrosine clone 4G10 (Upstate)) for 60 minutes, and washed with TBS-T (containing 25 mM Tris, 150 mM NaCl and 0.1% Tween-20) three times. Then, this PVDF membrane was shaken in 2 ml of a secondary antibody solution (containing 0.4% Block Ace and 2.7 μg/ml anti-mouse immunoglobulin rabbit polyclonal antibody labeled with FITC (DAKO)) for 60 minutes, and washed with TBS-T three times. This PVDF membrane was dried, and analyzed using an image analyzing device (Pharos FX system (Bio-Rad)) to detect fluorescent light.

4. Results

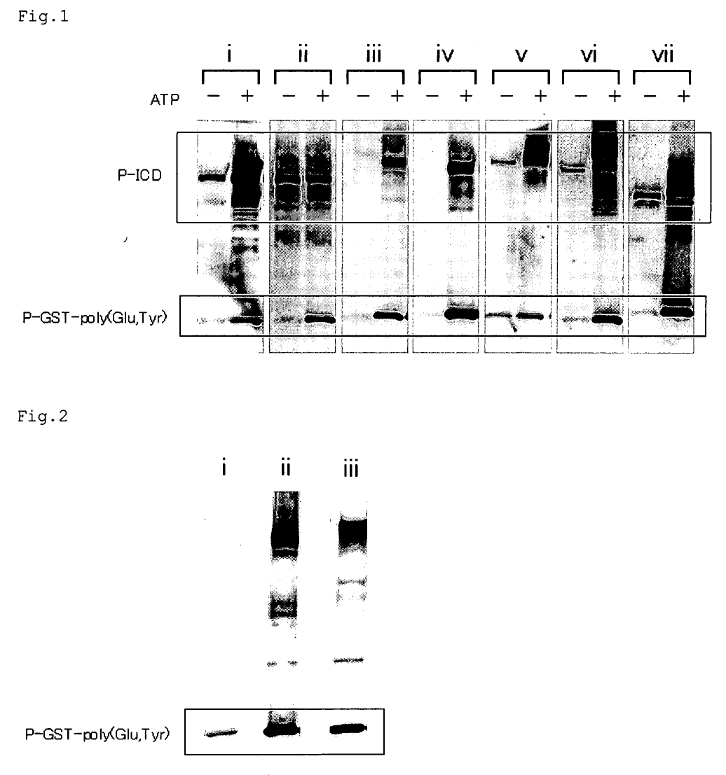

FIG. 1 shows the result of Western blotting. In the figure, i shows the result in the case of using PDGFR-β as tyrosine kinase, ii shows the result in the case of using VEGFR1, iii shows the result in the case of using VEGFR2, iv shows the result in the case of using HER1, v shows the result in the case of using HER2, vi shows the result in the case of using HER4, and vii shows the result in the case of using IGF1R. In addition, in each of i to vii, −is the result obtained from the SDS sample prepared using the substrate solution 2 not containing ATP. +is the result obtained from the SDS sample prepared using the substrate solution 1 containing ATP. P-ICD shows a position where an auto-phosphorylated tyrosine kinase appears. P-GST-poly (Glu, Tyr) shows a position where a phosphorylated GST-poly (Glu, Tyr) fusion protein appears.

In +of all of (i to vii) in FIG. 1, a single band was observed at a position where the phosphorylated GST-poly (Glu, Tyr) fusion protein appears. Thereby, it was found that the GST-poly (Glu, Tyr) fusion protein is phosphorylated by various kinds of tyrosine kinases.

In addition, in −of ii, iii, iv, vi and vii, a band is not observed at a position where the phosphorylated GST-poly (Glu, Tyr) fusion protein appears. The reason is thought as follows: ATP is not contained in a reaction solution in an enzyme reaction, and the GST-poly (Glu, Tyr) fusion protein is not phosphorylated. On the other hand, in −of i and v, a very faint band was observed at a position where the phosphorylated GST-poly (Glu, Tyr) fusion protein appears. The reason is thought as follows: an antibody used in detection bounds nonspecifically, or a minor amount of ATP was mixed in a product used in measurement.

### EXAMPLE 3

Detection of Phosphorylation of Fusion Protein by Western Blotting using Receptor Tyrosine Kinase Extracted from Cell Membrane

Herein, a receptor tyrosine kinase was extracted from a cell membrane of a cultured cell derived from a breast cancer, and the extracted receptor tyrosine kinase was used to phosphorylate GST-poly (Glu, Tyr) fusion protein prepared in Example 1. Then, the phosphorylated GST-poly (Glu, Tyr) fusion protein was detected by Western blotting.

1. Method of Preparing Reaction Sample

A cultured cell (MDA-MB231) derived from a breast cancer was cultured in a 225 cm² flask to 80% confluent (about 10⁷ cells) This cultured cell and 1 ml of a buffer 2 (containing 20 mM HEPES pH 7.4, 0.2% PI, 10% glycerol, 200 μM Na₃VO₄, and 50 mM NaF) were mixed. Then, a cell membrane of the cultured cell in the mixture was destroyed by pressuring with a pestle to obtain a cell solution. The resulting cell solution was centrifuged, and the supernatant was discarded. A precipitate and a buffer 3 (containing 20 mM HEPES pH 7.4, 1% NP40, 0.2% PI, 10% glycerol, 200 μM Na₃VO₄, and 50 mM NaF) were mixed. Then, a cell membrane in the mixture was solubilized by pressuring using a pestle, and this was centrifuged to recover the supernatant. This supernatant was used as a reaction sample in the following enzyme reaction. Similarly, from MDA-MB468 and SKBr3 which are cultured cells derived from a breast cancer, a reaction sample was prepared, respectively. Herein, a sample prepared from MDA-MB231 is designated as a reaction sample i, a sample prepared from MDA-MB468 is designated as a reaction sample ii, and a sample prepared from SKBr3 is designated as a reaction sample iii. Any of the reaction samples was prepared so that a protein concentration became 0.8 mg/ml.

2. Enzyme Reaction

25 μl of the reaction sample i obtained by the above method, and 25 μl of a substrate solution 3 (containing 20 Mm HEPES pH 7.4, 20 mM MnCl₂, 2 mM DTT, 1% NP40, 0.2% PI, 10% glycerol, 200 μM Na₃VO₄, 50 mM NaF, 100 μM ATP, and 5 μg GST-poly (Glu, Tyr) fusion protein) were mixed, and incubated at 25° C. for 30 minutes. To this reaction solution was added 25 μl of the SDS sample buffer used in Example 2, and this was boiled at 100° C. for 5 minutes to stop the enzyme reaction. The thus prepared solution is designated as an SDS sample i. Similarly, an SDS sample ii and an SDS sample iii were prepared from the reaction sample ii and the reaction sample iii, respectively.

3. Detection of Phosphorylated Fusion Protein by Western Blotting

In the same manner as in Example 2, the phosphorylated GST-poly (Glu, Tyr) fusion protein contained in the SDS samples (i to iii) was detected by Western blotting.

4. Results

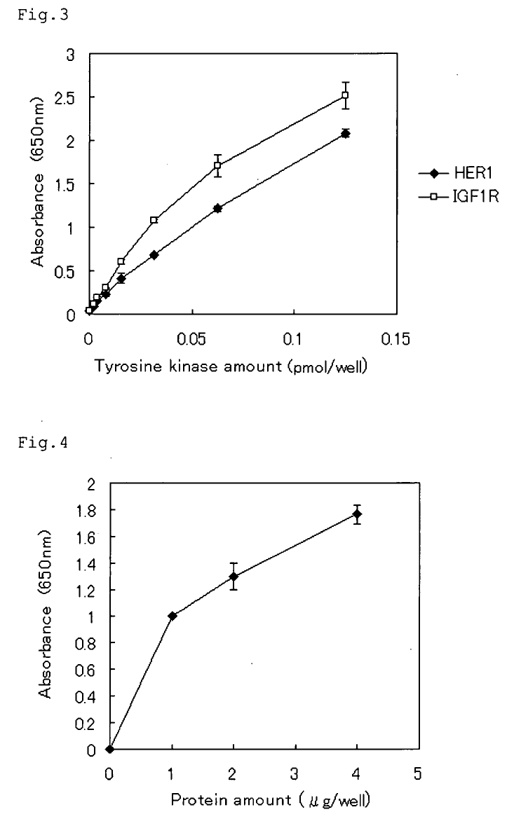

FIG. 2 shows the result of Western blotting. In the figure, i shows the result in the case of using a receptor tyrosine kinase extracted from a cell membrane of MDA-MB231, ii shows the result in the case of using a receptor tyrosine kinase extracted from a cell membrane of MDA-MB468, and iii shows the result in the case of using a receptor tyrosine kinase extracted from a cell membrane of SKBr3. P-GST-poly (Glu, Tyr) indicates a position where the phosphorylated GST-poly (Glu, Tyr) fusion protein appears.

In all of (i to iii), a single band was observed at a position where the phosphorylated GST-poly (Glu, Tyr) fusion protein appears. Thereby, it was found that the GST-poly (Glu, Tyr) fusion protein is phosphorylated by a tyrosine kinase present in a cell membrane. In addition, as shown in Example 2, the phosphorylated GST-poly (Glu, Tyr) fusion protein is phosphorylated by various kinds of tyrosine kinases. Thereby, it is thought that a band detected in this Example is the GST-poly (Glu, Tyr) fusion protein phosphorylated by various kinds of tyrosine kinases present in a cell membrane.

From the results of FIG. 1 of Example 2 and FIG. 2 of Example 3, it was found that the phosphorylated GST-poly (Glu, Tyr) fusion protein is separated as a single band from the other proteins in SDS-PAGE, and the separated GST-poly (Glu, Tyr) fusion protein is transferred to a PVDF membrane. Thereby, it was found that the GST-poly (Glu, Tyr) fusion protein can be applied to electrophoresis and Western blotting.

### EXAMPLE 4

Detection of Phosphorylation of Fusion Protein by ELISA using Intracellular Domain of Receptor Tyrosine Kinase

Herein, the GST-poly (Glu, Tyr) fusion protein prepared in Example 1 was phosphorylated using ICD of a commercially available receptor tyrosine kinase. Then, the phosphorylated GST-poly (Glu, Tyr) fusion protein was detected by ELISA.

1. Method of Preparing Reaction Sample

First, 50 μl of a buffer 4 (containing 20 mM HEPES pH 7.4, 10 mM MnCl₂, 1 mM DTT, 1% NP40, 0.2% PI, 10% glycerol, 200 μM Na₃VO₄, 50 mM NaF, 200 μM ATP) and 0.125 pmol of ICD of a commercially available receptor tyrosine kinase were mixed to prepare a reaction sample. Then, the prepared reaction sample was 2-fold diluted, 4-fold diluted, 8-fold diluted, 16-fold diluted, 32-fold diluted, and 64-fold diluted using the buffer 4. The thus prepared respective reaction samples (1-fold dilution to 64-fold dilution) were used in the following enzyme reaction. Further, the buffer 4 as a reaction sample containing no ICD was also used in the enzyme reaction. In addition, in the present Example, ICDs of HER1 and IGF1R (all from Cell Signaling Technology) used in Example 2 were used.

2. Binding of Fusion Protein to ELISA Plate

As the ELISA plate, a glutathione-coated plate (Reacti-Bind Clear Glutathione Coated Plates, 8-well Strip (PIERCE)) was used. First, each well of the plate was washed with TBS-T (containing 25 mM Tris, 150 mM NaCl and 0.05% Tween-20) three times. Then, into each well was added 50 μl of the substrate solution 3 (TBS containing 10 μg/ml GST-poly (Glu, Tyr) fusion protein) prepared in Example 1, and this was incubated at 25° C. for 1 hour while shaken slightly. After incubation, each well was washed with TBS-T two times, and further washed with 20 mM HEPES pH 7.4 (containing 0.05% Tween-20) once. Like this, the GST-poly (Glu, Tyr) fusion protein was made to bind to a surface of the well of the ELISA plate. This ELISA plate was used in the following enzyme reaction.

3. Enzyme Reaction and Detection of Phosphorylated Fusion Protein

Into separate wells of the ELISA plate was added 50 μl of each reaction solution, and this was incubated at 25° C. for about 30 minutes. After incubation, 100 μl of a reaction stop solution (TBS-T containing 1 mM EDTA) was added to each well, followed by washing with TBS-T three times. Then, each well was washed with 300 μl of a Starting Block T20 (TBS) Blocking Buffer (PIERCE) . An HRP-labeled primary antibody (p-Tyr (PY20), sc-508 HRP (SANTA Cruz Biotechnology)) was 1000-fold diluted with the Starting Block T20 (TBS) Blocking Buffer to obtain a primary antibody solution, and 100 μl of which was added into each well after washing, and incubated at 25° C. for about 1 hour and 30 minutes while shaken slightly. After incubation, each well was washed with TBS-T five times. Then, 150 μl of a TMB solution (3,3′,5,5′-Tetramethylbenzidine (TMB) Liquid Substrate System for ELISA (Sigma-Aldrich)) was added into each well after washing, a color was appropriately developed for 5 to 30 minutes while shielding light at room temperature, and an absorbance (650 nm) was measured with VersaMax (Molecular Device).

4. Results

FIG. 3 is a graph showing the result of ELISA. In the figure, an ordinate indicates an absorbance (650 nm), and an abscissa indicates a tyrosine kinase amount (pmol) per well.

In either case of HER1 and IGF1R, as the tyrosine kinase amount increased, a measured value was increased. Thereby, it was found that phosphorylation of the GST-poly (Glu, Tyr) fusion protein can be detected in ELISA.

### EXAMPLE 5

Detection of Phosphorylation of Fusion Protein by ELISA using Receptor Tyrosine Kinase Extracted from Cell Membrane

Herein, the GST-poly (Glu, Tyr) fusion protein prepared in Example 1 was phosphorylated using a receptor tyrosine kinase extracted from a cell membrane of a cultured cell derived from a breast cancer. Then, the phosphorylated GST-poly (Glu, Tyr) fusion protein was detected by ELISA.

1. Method of Preparing Reaction Sample

A cultured cell (MDA-MB468) derived from a breast cancer was cultured in a 225 cm² flask to 80% confluent (about 10⁷ cells) This cultured cell and 1 ml of the buffer 2 used in Example 3 were mixed. Then, a cell membrane of the cultured cell in the mixture was destroyed by pressurizing using a pestle to obtain a cell solution. The resulting cell solution was centrifuged, and the supernatant was discarded. A precipitate and the buffer 3 used in Example 3 were mixed. Then, a cell membrane in the mixture was solubilized by pressurizing using a pestle, and this was centrifuged to recover the supernatant. This supernatant was diluted with the buffer 4, and reaction samples having a protein concentration of 0.02 mg/ml, 0.04 mg/ml and 0.08 mg/ml were prepared, respectively. Each reaction sample thus prepared was used in the following enzyme reaction. In addition, as a reaction sample not containing a protein, the buffer 3 was used in the following enzyme reaction.

2. Binding of Fusion Protein to ELISA Plate

In the same manner as in Example 4, the GST-poly (Glu, Tyr) fusion protein was bound to a surface of a well of the ELISA plate. Then, this ELISA plate was used in the following enzyme reaction.

3. Enzyme Reaction and Detection of Phosphorylated Fusion Protein

In the same manner as in Example 4, the GST-poly (Glu, Tyr) fusion protein phosphorylated by a tyrosine kinase in each reaction sample was detected.

4. Results

FIG. 4 is a graph showing the result of ELISA. In the figure, an ordinate indicates an absorbance (650 nm), and an abscissa indicates a protein amount (μg) per well (50 μl).

As the protein amount increased, that is, the tyrosine kinase amount increased, a measured value was increased. From this, it was found that the GST-poly (Glu, Tyr) fusion protein is phosphorylated by a tyrosine kinase present in a cell membrane. In addition, as shown in Example 2 and Example 4, the GST-poly (Glu, Tyr) fusion protein is phosphorylated by various kinds of tyrosine kinases. Thereby, it is thought that the measured value detected in the present Example detected the GST-poly (Glu, Tyr) fusion protein phosphorylated by various kinds of tyrosine kinases present in a cell membrane.

### EXAMPLE 6

Detection of Fusion Protein Adsorbed onto Slot Blot Membrane

Herein, in accordance with a procedure of slot blotting, the GST-poly (Glu, Tyr) fusion protein prepared in Example 1 was adsorbed onto a membrane, and the GST-poly (Glu, Tyr) fusion protein adsorbed onto the membrane was detected.

1. Preparation of Slot Blotting Sample

The GST-poly (Glu, Tyr) fusion protein obtained in Example 1 was dissolved in TBS (25 mM Tris pH 7.4, 150 mM NaCl) to concentrations of 10 μg/ml, 5 μg/ml, 2.5 μg/ml, 1.25 μg/ml, 0.625 μg/ml and 0 μg/ml, and the prepared solutions were used as slot blotting samples. Further, for comparison, a commercially available synthetic peptide (STG200 (CHEMICON)) was dissolved in TBS (25 mM Tris pH 7.4, 150 mM NaCl) to concentrations of 10 μg/ml, 5 μg/ml, 2.5 μg/ml, 1.25 μg/ml, 0.625 μg/ml and 0 μg/ml, and the solutions were used as slot blotting samples. The commercially available synthetic peptide (STG200) used herein is a synthetic peptide obtained by binding biotin to a polypeptide in which an amino acid sequence of SEQ ID No.1 (Glu-Glu-Glu-Glu-Tyr) is repeated plural times.

2. Adsorption onto Membrane

Each 100 μl of the slot blotting sample was added in a separate well of a slot blotter (BIO-DOT SF (Bio-Rad)) having a PVDF membrane (Immobilon-PSQ 0.2 μm pore size (Millipore)) on a bottom surface. The slot blotting sample was sucked from a back side of the PVDF membrane using a vacuum pump, and a protein contained in each slot blotting sample was adsorbed onto the PVDF membrane. Thereafter, the PVDF membrane was removed from the slot blotter, and the protein in the PVDF membrane was stained with a CBB staining solution (Page Blue 83 staining solution (CBB-R250) (Daiichi Pure Chemicals Co., Ltd.)).

3. Results

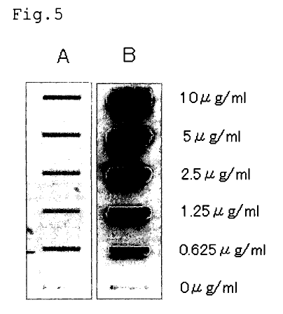

FIG. 5 shows a membrane of slot blotting after CBB staining. A is the result of blotting of a sample containing the GST-poly (Glu, Tyr) fusion protein. B is the result of blotting of a sample containing the commercially available synthetic peptide (STG200). Further, each band of A is different in a concentration of the GST-poly (Glu, Tyr) fusion protein contained in a blotted sample, and the concentrations are 10 μg/ml, 5 μg/ml, 2.5 μg/ml, 1.25 μg/ml, 0.625 μg/ml and 0 μg/ml from the top. Similarly, each band of B is different in a concentration of the synthetic peptide (STG200 ) in a sample, and the concentrations are 10 μg/ml, 5 μg/ml, 2.5 μg/ml, 1.25 μg/ml, 0.625 μg/ml and 0 μg/ml from the top.

In A, a band reflecting a shape of a well of the slot blotter was detected. In addition, a concentration of each band was increased depending on a concentration of the GST-poly (Glu, Tyr) fusion protein in a sample. Thereby, it was found that the GST-poly (Glu, Tyr) fusion protein was effectively adsorbed onto the PVDF membrane in slot blotting in a concentration-dependent manner. In addition, it was found that the GST-poly (Glu, Tyr) fusion protein can be applied to slot blotting. On the other hand, in a band detected in B, a shape of the band was deformed as a concentration of the fusion protein in a sample was increased. Particularly, regarding bands of 10 μg/ml, 5 μg/ml, 2.5 μg/ml, and 1.25 μg/ml, a concentration on an internal side was lower as compared with a concentration on an edge of the band. In slot blotting, when quantitative measurement (e.g. measurement of activity value of tyrosine kinase) is performed using a band in such a state, a precise measurement result cannot be obtained.

What is claimed is:

1. A tyrosine kinase substrate comprising a fusion protein which comprises a protein for labeling and a specific peptide fused with the protein for labeling,
wherein the specific peptide has an amino acid sequence including a glutamic acid residue and a tyrosine residue.

2. The substrate according to claim 1, wherein the substrate is capable of being phosphorylated by a plurality of kinds of tyrosine kinases.

3. The substrate according to claim 1, wherein the amino acid sequence is at least one selected from the group consisting of:
an amino acid sequence I consisting of a glutamic acid residue and a tyrosine residue, in which a ratio of the glutamic acid residue and the tyrosine residue is 4:1,
an amino acid sequence II consisting of a glutamic acid residue and a tyrosine residue, in which a ratio of the glutamic acid residue and the tyrosine residue is 1:1,
an amino acid sequence III consisting of a glutamic acid residue, a tyrosine residue, and an alanine residue, in which a ratio of the glutamic acid residue, the alanine residue and the tyrosine residue is 6:1:3,
an amino acid sequence IV consisting of a glutamic acid residue, a tyrosine residue, and an alanine residue, in which a ratio of the glutamic acid residue, the alanine residue and the tyrosine residue is 1:1:1, and
an amino acid sequence V consisting of a glutamic acid residue, a tyrosine residue, an alanine residue, and a lysine residue, in which a ratio of the glutamic acid residue, the tyrosine residue, the alanine residue, and the lysine residue is 2:1:6:5.

4. The substrate according to claim 3, wherein the amino acid sequence I is an amino acid sequence in which a sequence consisting of four glutamic acid residues and one tyrosine residue is repeated two or more times.

5. The substrate according to claim 3, wherein the amino acid sequence II is an amino acid sequence in which a sequence consisting of one glutamic acid residue and one tyrosine residue is repeated two or more times.

6. The substrate according to claim 3, wherein the amino acid sequence III is an amino acid sequence in which a sequence consisting of six glutamic acid residues, one tyrosine residue and three alanine residues is repeated two or more times.

7. The substrate according to claim 3, wherein the amino acid sequence IV is an amino acid sequence in which a sequence consisting of one glutamic acid residue, one tyrosine residue and one alanine residue is repeated two or more times.

8. The substrate according to claim 3, wherein the amino acid sequence V is an amino acid sequence in which a sequence consisting of two glutamic acid residues, one tyrosine residue, six alanine residues and five lysine residues is repeated two or more times.

9. The substrate according to claim 1, wherein the tyrosine kinase is a receptor tyrosine kinase.

10. The substrate according to claim 9, wherein the receptor tyrosine kinase comprises at least an insulin-like growth factor receptor (IGFR), a platelet-derived growth factor receptor (PDGFR), a human epithelial growth factor receptor (HER) and a vascular endothelial growth factor receptor (VEGFR).

11. The substrate according to claim 1, wherein the protein for labeling has a molecular weight of 10 kDa or more.

12. The substrate according to claim 11, wherein the protein for labeling has a molecular weight of 100 kDa or less.

13. The substrate according to claim 1, wherein the protein for labeling is an affinity tag.

14. The substrate according to claim 13, wherein the affinity tag has a molecular weight of 10 kDa or more.

15. The substrate according to claim 13, wherein the affinity tag is at least one selected from the group consisting of glutathione-S-transferase, maltose binding protein, avidin and streptavidin.

16. The substrate according to claim 1, wherein the specific peptide is fused with the protein for labeling via one or more amino acid residues.

17. A method for measuring the activity of a tyrosine kinase, comprising steps of:
contacting the tyrosine kinase with a tyrosine kinase substrate and a phosphate group donor so that phosphorylate the substrate, wherein
the substrate comprises a fusion protein which comprises a protein for labeling and a specific peptide fused with the protein for labeling, and
the specific peptide has an amino acid sequence including a glutamic acid residue and a tyrosine residue;
detecting the phosphorylated substrate; and
measuring the activity of the tyrosine kinase based on the result of detection of the phosphorylated substrate.

18. The method according to claim 17, wherein the phosphate group donor is at least one selected from the group consisting of adenosine triphosphate (ATP), adenosine 5′-O-(3-thiotriphosphate) (ATP-γS), 32P-labeled adenosine 5′-O-(3-triphosphate) (γ-[32P]-ATP), adenosine diphosphate (ADP) and adenosine monophosphate (AMP).

19. The method according to claim 17, further comprising a step of subjecting the phosphorylated substrate to electrophoresis.

20. The method according to claim 17, further comprising a step of adsorbing the phosphorylated substrate onto a membrane.
